(19) United States
(12) Patent Application Publication
(10) Pub. No.: US 2024/0209122 A1
(43) Pub. Date: Jun. 27, 2024

(54) NONENYL SUCCINIC ANHYDRIDE MODIFIED STARCH, PREPARATION METHOD THEREFOR, AND APPLICATION THEREOF

(71) Applicant: Guantian LI, Auckland (NZ)

(72) Inventors: Guantian LI, Auckland (NZ); Fan ZHU, Auckland (NZ)

(21) Appl. No.: 18/553,615

(22) PCT Filed: Apr. 1, 2021
(86) PCT No.: PCT/CN2021/084993

§ 371 Date: Oct. 2, 2023

(51) Int. Cl. C08B 31/04; A23L 29/219; A61K 8/73; A61K 47/36

(52) U.S. Cl. CPC C08B 31/04; A23L 29/219; A61K 8/732; A61K 47/36

(57) ABSTRACT

Nonenyl succinic anhydride modified starch and a preparation method therefor, and a Pickering emulsion comprising nonenyl succinic anhydride modified starch. The chemically modified starch, as a particulate emulsifier, has excellent emulsification performance and wide application prospects.

### FIELD OF THE INVENTION

The present invention belongs to the field of modified starches, and specifically relates to nonenyl succinic anhydride modified starch and its preparation method and application.

### BACKGROUND OF THE INVENTION

Pickering emulsion is a novel type of emulsion system stabilized by solid particles rather than small molecule surfactants. It has broad application prospects in fields such as food, cosmetics, biomedicine, papermaking, coating, and petroleum exploration. Starch, as a natural biomaterial with abundant sources, low price, degradability, and good biocompatibility, has been used as particulate emulsifier in Pickering emulsions. However, natural starch contains a large amount of hydrophilic hydroxyl groups. When natural starch is used alone as particulate emulsifier, its emulsification effect is generally poor, and the emulsion stability is unsatisfactory. Previous researchers have attempted to improve the emulsification properties of starch through hydrophobic modification. Common starch modifiers include octenyl succinic anhydride (OSA) and dodecenyl succinic anhydride (DDSA). OSA modified starch has been extensively studied. However, there is still a need to develop a new modified starch to overcome the deficiencies of OSA modified starch or DDSA modified starch.

### SUMMARY OF THE INVENTION

In order to overcome the deficiencies of OSA modified starch or DDSA modified starch, the present disclosure provides a nonenyl succinic anhydride (NSA) modified starch. Compared with DDSA modified starch, the modified starch can reach a more suitable degree of substitution. Under the same degree of substitution, NSA modified starch has better emulsification performance than OSA modified starch. Another aspect of the present disclosure provides the use of NSA modified starch as an emulsifier. Yet another aspect of the present disclosure provides a Pickering emulsion comprising NSA modified starch. The NSA modified starch of the present disclosure exhibits excellent emulsification performance as an emulsifier for Pickering emulsions.

### BRIEF DESCRIPTION OF THE DRAWINGS

In order to clearly illustrate the technical solutions of the examples of the present invention, a brief introduction to the figures of the examples will be given as follows. Apparently, the figures described below are only related to some examples of the present invention, rather than limitations on the present invention.

FIG. 1 shows the SEM images of unmodified quinoa starch and NSA modified quinoa starches with different degrees of substitution in the examples of the present disclosure: a) unmodified quinoa starch; b) NSA 1 (DS 0.0080); c) NSA 2 (DS 0.0175); d) NSA 3 (DS 0.0359); and e) NSA 4 (DS 0.0548).

FIG. 2 shows Fourier Transform Infrared Spectroscopy (FTIR) spectra of unmodified quinoa starch and NSA modified quinoa starch with different degrees of substitution in the examples of the present disclosure.

FIG. 3 shows particle size distributions of unmodified quinoa starch and NSA modified quinoa starch with different degrees of substitution in the examples of the present disclosure.

FIG. 4 shows particle size distributions of droplets in Pickering emulsions stabilized by unmodified/NSA modified quinoa starch in the examples of the present disclosure during storage: a) unmodified quinoa starch; b) NSA 1 (DS 0.0080); c) NSA 2 (DS 0.0175); d) NSA 3 (DS 0.0359); and e) NSA 4 (DS 0.0548).

### DETAILED DESCRIPTION OF THE INVENTION

In order to clarify the objectives, technical solutions, and advantages of the embodiments of the present disclosure, the technical solutions in the embodiments of the present disclosure will be described below in conjunction with the figures of the embodiments of the present disclosure. Apparently, the described embodiments are merely a part of the embodiments of the present disclosure, rather than all of the embodiments. All other embodiments obtained by a person with ordinary skill in the art based on the embodiments of the present disclosure described without creative efforts shall fall within the protection scope of the present invention.

It should be understood that all or any of the embodiments of the present invention can be combined with each other as long as no conflict exists. The present invention includes additional embodiments obtained from such combinations.

All publications and patents mentioned in the present disclosure are hereby fully incorporated by reference into the present disclosure in their entireties. In case of any conflict between the usage or terminology in any publication or patent incorporated by reference and the usage or terminology in the present disclosure, the usage and terminology in the present disclosure shall prevail.

Unless otherwise specified, all technical terms and scientific terms used herein have the ordinary meaning commonly understood by a person skilled in the art to which the subject matter belongs. If there are multiple definitions for a term, the definition in this document shall prevail.

The section headings used herein are for organizational purposes only and shall not be construed as limiting the subject matter described.

Unless otherwise noted, whenever any kind of scope (e.g., degree of substitution) is disclosed or claimed, it is intended individually to disclose or claim any possible values that the scope can reasonably encompass, including the end points and any sub-range therebetween. For example, a degree of substitution of 0.0050 to 0.023 is intended to include 0.0050 and 0.023 individually, as well as sub-ranges 0.0050-0.010, 0.0080-0.012, 0.010-0.015, 0.010-0.020, 0.015-0.020, 0.015-0.023, etc.

The terms “comprising”, “containing”, or “including” and the like used in the present disclosure refer to the elements present before these terms covering the elements listed after these terms and their equivalents, while not excluding undisclosed elements. The terms “containing” or “including (comprising)” used herein may be open, semi-closed, or closed. In other words, the terms also include “consisting essentially of”, or “consisting of”.

It should be understood that the singular forms (such as “a”) used in the present disclosure can include plural referents unless otherwise specified.

The reagents and raw materials used in the present disclosure are commercially available or can be prepared by conventional chemical synthesis methods. Unless otherwise specified, conventional techniques or conditions in the art, or instructions on product manuals can be followed for specifics not explicitly described in the following examples. Reagents or instruments without indicated manufacturers are conventional products commercially available.

The starch for modification may be derived from cereals, tubers, roots, legumes, and fruits, such as barley, wheat, quinoa, oats, buckwheat, rye, sorghum, corn, potato, sweet potato, cassava, chickpea, mung bean, pea, banana, plantain, etc. In one embodiment, small granular starch is preferred for NSA modification. In a preferred embodiment, the starch for modification is derived from quinoa. Quinoa starches are small granular starches with most granule sizes distributed between 0.4 μm and 2.0 μm, smaller than most other plant-based starches such as corn starch and potato starch. Modified quinoa starch as a particulate emulsifier has stronger capability of emulsion stabilization. Larger granular starches such as corn starch (granule size about 20 μm) and potato starch (granule size about 50 μm) can be made into smaller particles by processing such as grinding and sieving, and then used for NSA modification.

The NSA modified starch of the present disclosure can be prepared by esterification reaction between starch and NSA. The reaction is shown below.

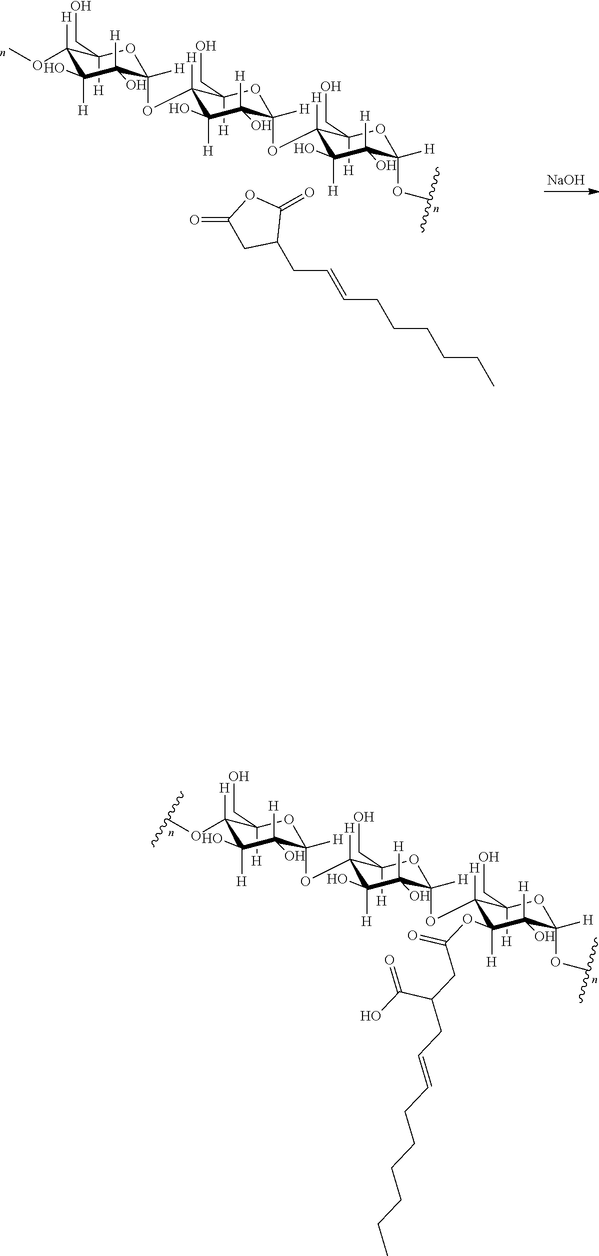

In one embodiment, the preparation method of nonenyl succinic anhydride modified starch includes the following steps:
1) mixing starch with water to form a suspension;
2) slowly adding NSA to the suspension described in step 1) with stirring;
3) adding alkali or acid to control the pH of the suspension; and
4) obtaining NSA modified starch by centrifugation.

In a preferred embodiment, NSA is first mixed with a solvent to form a mixture before adding to the suspension described in step 1). The solvent is selected from water, ethanol, acetone or mixtures thereof.

In a more preferred embodiment, the solvent is water, and a small amount of surfactant is added in the mixture of NSA and the solvent. The surfactant is selected from sodium dodecyl sulfate (SDS), sodium hexadecyl sulfate, sodium octadecyl sulfate or mixtures thereof.

In one embodiment, the preparation method of NSA modified starch is as follows: starch is mixed with water to form a suspension; NSA is slowly added to the suspension with stirring; during the reaction with stirring, the pH is controlled to weak alkaline (e.g. 8.0-9.0) by adding alkali such as 2-20 wt % NaOH. After reacting for a period of time (e.g. 1-6 h), the pH is adjusted (e.g. 6.5-7.0) by adding diluted acid such as 2-10 wt % hydrochloric acid to terminate the reaction. NSA modified starch is recovered by centrifugation. The reaction with stirring can be carried out at room temperature or in a water bath, for example, in a water bath at 30° C.

The success of NSA modification can be verified by FTIR. Two peaks related to asymmetric stretching vibration of RCOO— groups and stretching vibration of C—O groups in carbonyl confirm the successful esterification reaction between starch and NSA.

The degree of substitution refers to the ratio of the number of esterified hydroxyl groups to the total number of glucose residues in the modified starch. By controlling the mass ratio of NSA to starch in the esterification reaction, the degree of substitution of the modified starch can be adjusted. The degree of substitution can be determined according to the method in Bao, J., Xing, J., Phillips, D. L., & Corke, H. (2003). Physical properties of octenyl succinic anhydride modified rice, wheat, and potato starches. Journal of Agricultural and Food Chemistry, 51, 2283-2287. In one embodiment, the degree of substitution of the NSA modified starch does not exceed 0.10. In a preferred embodiment, the degree of substitution of the NSA modified starch does not exceed 0.060. In a more preferred embodiment, the degree of substitution of the NSA modified starch does not exceed 0.023. For example, in one embodiment, the degree of substitution of the NSA modified starch is 0.0050 to 0.023, 0.0060 to 0.023, 0.0070 to 0.023, 0.0080 to 0.023, 0.0090 to 0.023, 0.010 to 0.023, 0.011 to 0.023, 0.012 to 0.023, 0.013 to 0.023, 0.014 to 0.023, 0.015 to 0.023, 0.016 to 0.023, 0.017 to 0.023, 0.018 to 0.023, 0.019 to 0.023, 0.020 to 0.023, 0.021 to 0.023, or 0.022 to 0.023. For example, in another embodiment, the degree of substitution of the NSA modified starch is 0.0050 to 0.020, 0.0060 to 0.020, 0.0070 to 0.020, 0.0080 to 0.020, 0.0090 to 0.020, 0.010 to 0.020, 0.011 to 0.020, 0.012 to 0.020, 0.013 to 0.020, 0.014 to 0.020, 0.015 to 0.020, 0.016 to 0.020, 0.017 to 0.020, 0.018 to 0.020, or 0.019 to 0.020. For example, in one embodiment, the degree of substitution of the NSA modified starch is 0.010 to 0.020. For example, in one embodiment, the degree of substitution of the NSA modified starch is 0.015 to 0.023.

In one embodiment, the change in mass-moment mean diameter or De Brouckere mean diameter D[4,3] of starch before and after NSA modification does not exceed 25%, for example no higher than 24%, no higher than 23%, no higher than 22%, no higher than 21%, no higher than 20%, no higher than 19%, no higher than 18%, no higher than 17%, no higher than 16%, no higher than 15%, no higher than 14%, no higher than 13%, no higher than 12%, no higher than 11%, no higher than 10%, or no higher than 9%. Typically, the mass-moment mean diameter or De Brouckere mean diameter D[4,3] of starch decreases after NSA modification.

In one embodiment, the De Brouckere mean diameter D[4,3] of NSA modified starch is less than or equal to 5.0 μm, for example, less than or equal to 4.0 μm, less than or equal to 3.0 μm, less than or equal to 2.5 μm, less than or equal to 2.0 μm, or less than or equal to 1.8 μm. In one embodiment, the De Brouckere mean diameter D[4,3] of NSA modified starch is greater than or equal to 0.10 μm, for example, greater than or equal to 0.20 μm, greater than or equal to 0.30 μm, greater than or equal to 0.40 μm, greater than or equal to 0.50 μm, greater than or equal to 0.60 μm, greater than or equal to 0.70 μm, greater than or equal to 0.80 μm, greater than or equal to 0.90 μm, greater than or equal to 1.0 μm, greater than or equal to 1.1 μm, greater than or equal to 1.2 μm, greater than or equal to 1.3 μm, greater than or equal to 1.4 μm, greater than or equal to 1.5 μm, greater than or equal to 1.6 μm, or greater than or equal to 1.7 μm. In one embodiment, the De Brouckere mean diameter D[4,3] of NSA modified starch is greater than or equal to 0.10 μm and less than or equal to 3.0 μm. In one embodiment, the De Brouckere mean diameter D[4,3] of NSA modified starch is greater than or equal to 0.50 μm and less than or equal to 2.0 μm.

The NSA modified starch of the present disclosure introduces hydrophilic carboxylic acid groups and hydrophobic long alkenyl chains into the starch. It is believed that during emulsion preparation, the hydrophilic carboxylic acid groups extend into the aqueous phase, the hydrophobic nonenyl long chains extend into the oil phase, and the complex polysaccharide long chains unfold at the oil-water interface to form a continuous, dense, and unbreakable interfacial membrane. The emulsification performance of the NSA modified starch of the present disclosure is significantly improved compared to the unmodified starch. The NSA modified starch of the present disclosure can be used as a particulate emulsifier for emulsification of foods, pharmaceuticals, or cosmetics at suitable degrees of substitution. Of course, the NSA modified starch of the present disclosure as a particulate emulsifier may also be used in other fields such as coatings, petroleum exploration, paper making, etc.

The NSA modified starch of the present disclosure can be used as a particulate emulsifier for the preparation of Pickering emulsions. In one embodiment, the present disclosure provides an O/W Pickering emulsion containing the aforementioned NSA modified starch as a particulate emulsifier. In another embodiment, the present disclosure provides a W/O Pickering emulsion containing the aforementioned NSA modified starch as a particulate emulsifier.

The O/W Pickering emulsion can be prepared by the following method:
1) mixing the aqueous phase to be processed with the aforementioned NSA modified starch using a high speed homogenizer, for example at a speed of 5000-25000 rpm; and
2) slowly adding the oil phase to the aqueous phase, and continuing high-speed shear homogenization to obtain the emulsion.

The W/O Pickering emulsion can be prepared by the following method:
1) mixing the oil phase to be processed with the aforementioned NSA modified starch using a high speed homogenizer, for example at a speed of 5000-25000 rpm;
2) slowly adding the aqueous phase to the oil phase and continuing high-speed shear homogenization to obtain the emulsion.

The NSA modified starch of the present disclosure exhibits excellent emulsification performance as a particulate emulsifier for Pickering emulsions.

The present invention will be further described below with reference to specific examples. It should be noted that these examples are illustrative only and in no way limit the present invention.

(2-Nonaen-1-yl) succinic anhydride (NSA, purity ≥85%) was purchased from Sigma-Aldrich Chemical Co. (St. Louis, Missouri, USA). All other chemicals were analytical grade.

The following illustrates NSA modified starch and its application with quinoa starch as an example.

### Quinoa Starch for Modification

The quinoa starch “S1” in Table 1 of Li, G., Wang, S., & Zhu, F. (2016). Physicochemical properties of quinoa starch. Carbohydrate Polymers, 137, 328-338 was used. Quinoa starch was isolated from quinoa seeds (brand: Fresh Produce Be Fresh Quinoa; Countdown supermarket; Auckland, New Zealand). As estimated by concanavalin A precipitation, the content of amylose in quinoa starch was 10.9%. The prepared quinoa starch was used for NSA modification.

### Preparation of NSA Modified Quinoa Starch:

Unmodified quinoa starch (25 g, dry basis) was mixed with 150 mL of deionized water in a three-necked flask to form a suspension. NSA was mixed with water to 20% (w/v). After adding a small amount of surfactant (about 50 mg of SDS), the mixture was vigorously shaken to form an emulsion. The emulsion was then added dropwise to the three-necked flask under continuous stirring. The pH of the suspension was maintained between 8.0-9.0 using a diluted NaOH solution. The reaction temperature was kept at 25° C. After the pH became stable, the pH of the suspension was adjusted to 6.5-7.0 with dilute HCl solution. The modified quinoa starch was recovered by centrifugation at 4000×g for 20 minutes. The starch cake was washed once with ethanol, twice with acetone, then dried in a 40° C. air oven for 48 hours to obtain the desired chemically modified starch.

### Example NSA1

25 g of quinoa starch and 0.50 g of NSA were used according to the above method to produce a modified quinoa starch with a degree of substitution of 0.0080.

### Example NSA2

25 g of quinoa starch and 1.0 g of NSA were used according to the above method to produce a modified quinoa starch with a degree of substitution of 0.0175.

### Example NSA3

25 g of quinoa starch and 2.0 g of NSA were used according to the above method to produce a modified quinoa starch with a degree of substitution of 0.0359.

### Example NSA4

25 g of quinoa starch and 3.0 g of NSA were used according to the above method to produce a modified quinoa starch with a degree of substitution of 0.0548.

### Determination of Degree of Substitution (DS)

The determination of degree of substitution (DS) was modified based on Bao, J., Xing, J., Phillips, D. L., & Corke, H. (2003). Physical properties of octenyl succinic anhydride modified rice, wheat, and potato starches. Journal of Agricultural and Food Chemistry, 51, 2283-2287. Briefly, modified and unmodified starches (2.0 g, dry basis) were suspended in 50 mL of distilled water in a conical flask. The starch was gelatinized in a boiling water bath for 30 min, then cooled under stirring at room temperature. Sodium hydroxide solution (25 mL, 0.50 M) was added to the conical flask, which was then kept shaking overnight. The resulting suspension was titrated with HCl solution (0.34 M) to pH 7 using phenolphthalein as an indicator. The amounts of HCl consumed for unmodified and chemically modified starches were recorded, and the DS values were calculated according to Wurzburg, O. B. (1964) Starch derivatives and modification. In Methods in Carbohydrate Chemistry, IV; Whistler, R. L, Ed.; Academic Press: New York, pp 286-288.

$$\begin{matrix}NSA : \\ NSA \% = \frac{( {V}_{blank} - {V}_{sample} ) \times M \times 0.224 \times 100}{W}\end{matrix} \qquad ( 1 )$$

$$DS = \frac{162 \times NSA \%}{22400 - ( 223 \times NSA \% )} \qquad ( 2 )$$

$$\begin{matrix}OSA : \\ OSA \% = \frac{( {V}_{blank} - {V}_{sample} ) \times M \times 0.21 \times 100}{W}\end{matrix} \qquad ( 3 )$$

$$DS = \frac{162 \times OSA \%}{21000 - ( 209 \times OSA \% )} \qquad ( 4 )$$

$$\begin{matrix}DDSA : \\ DDSA \% = \frac{( {V}_{blank} - {V}_{sample} ) \times M \times 0.266 \times 100}{W}\end{matrix} \qquad ( 5 )$$

$$DS = \frac{162 \times DDSA \%}{26600 - ( 265 \times DDSA \% )} \qquad ( 6 )$$

Where, V_{blank} and Vₛₐₘₚₗₑ are the volumes of HCl consumed for titrating the blank sample (unmodified starch) and chemically modified sample, respectively (mL); M is the molar concentration of HCl (mol/L); W is the sample weight (g).

The small granules of quinoa starch (about 2 μm) allow high contact area between the modifier and starch, resulting in high degrees of substitution. According to FDA regulations, the maximum allowed OSA for OSA modified starch in food is 3% (w/w in starch, DS of about 0.023). In the examples, the DS of the two NSA samples (NSA 1 and 2) were within this standard, while the higher DS of the other two samples (NSA 3 and 4) make them suitable for non-food applications.

OSA modified quinoa starch and DDSA modified quinoa starch were prepared similarly to the preparation method of the aforementioned NSA modified quinoa starch. Their DS were measured according to the above method. The DS of DDSA modified quinoa starch was much lower, only 0.0023 to 0.0095. The lower DS of DDSA modified starch may be because DDSA has a longer alkenyl chain with lower solubility in aqueous environment and has solid nature at room temperature. These properties may limit the contact between DDSA and starch granules, resulting in decreased DS.

### Morphology

Scanning electron microscope (SEM) images of unmodified and NSA modified quinoa starches were obtained using a Hitachi S-3400N scanning electron microscope (Tokyo, Japan). The images were taken at an accelerating voltage of 5 kV. The magnification was 10.0 k.

FIG. 1 shows the scanning electron microscope images of the example samples, wherein a is unmodified quinoa starch granules; b is NSA modified quinoa starch with DS of 0.0080; c is NSA modified quinoa starch with DS of 0.0175; d is NSA modified quinoa starch with DS of 0.0359; e is NSA modified quinoa starch with DS of 0.0548.

### FTIR Analysis

FTIR spectra were measured in the wavelength range of 400 cm⁻¹ to 4000 cm⁻¹ using a Bruker Vertex 70 spectrometer (Bruker Optik GmbH, Ettlingen, Germany). The air spectrum was used as blank, and each sample spectrum was averaged over 64 independent scans.

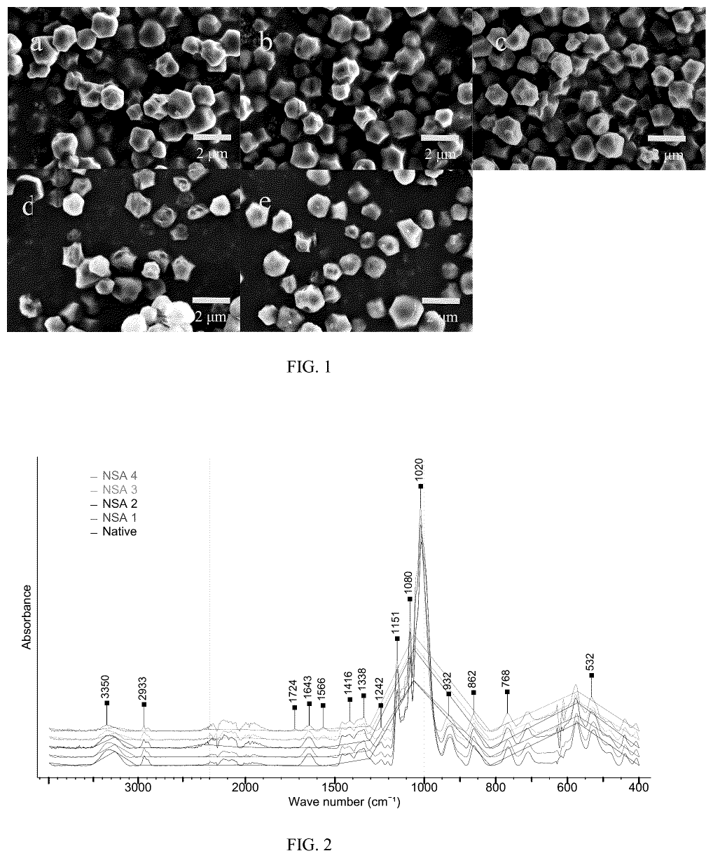

FIG. 2 shows the FTIR analysis results of unmodified and NSA modified quinoa starches in the examples. After the NSA modification of starch, two peaks began to appear at around 1566 cm⁻¹ and 1724 cm⁻¹, and the peak areas of these two peaks increased with increasing degree of substitution. The peak around 1566 cm⁻¹ is related to asymmetric stretching vibration of RCOO— groups, indicating the presence of esterification and monoester formation. The peak at 1724 cm⁻¹ is related to stretching vibration of C—O groups in carbonyl. These two peaks confirm the successful esterification between starch and NSA.

### Particle Size Distribution

Particle size distribution was determined by laser light scattering. The particle size distribution of unmodified and NSA modified quinoa starches was measured using a Mastersizer 2000 particle size analyzer (Malvern Instruments, Worcestershire, UK) following the steps as described in Li, G., & Zhu, F. (2017) Amylopectin molecular structure in relation to physicochemical properties of quinoa starch, Carbohydrate Polymers, 164, 396-402. Starch suspensions (1%, w/w) were stirred overnight at 300 rpm, then slowly added to the sample dispersion unit filled with water until the obscuration range was between 10% and 20%. The mixing speed of the dispersion unit was maintained at 2100 rpm. The refractive index of the particles, absorption index of the particles, and refractive index of the dispersant were defined as 1.5, 0, and 1.33, respectively. The number mean diameter d[n,0.5] (diameter based on the median position of the number-based size distribution), mass-moment mean diameter or De Brouckere mean diameter D[4,3], and surface area moment mean diameter or Sauter mean diameter D[3,2] were recorded. Span (measure of distribution width), uniformity (measure of deviation from the median), and specific surface area (SSA, surface area per unit weight) were also calculated.

TABLE 1
Degree of substitution and particle size of NSA modified quinoa starches.

| | Degree of | D [3, 2] | D [4, 3] | | |
|---|---|---|---|---|---|
| Sample | substitution | (μm) | (μm) | Span | Uniformity |
| Unmodified | — | 1.76 ± 0.00 a | 1.96 ± 0.00 a | 0.75 ± 0.00 a | 0.24 ± 0.00 a |
| NSA 1 | 0.0080 ± 0.0008 d | 1.61 ± 0.03 c | 1.78 ± 0.09 cd | 0.71 ± 0.05 b | 0.22 ± 0.02 b |
| NSA 2 | 0.0175 ± 0.0016 c | 1.57 ± 0.02 d | 1.73 ± 0.08 d | 0.71 ± 0.05 b | 0.23 ± 0.02 b |
| NSA 3 | 0.0359 ± 0.0016 b | 1.61 ± 0.02 c | 1.81 ± 0.03 bc | 0.74 ± 0.00 ab | 0.24 ± 0.00 a |
| NSA 4 | 0.0548 ± 0.0032 a | 1.72 ± 0.00 b | 1.86 ± 0.00 b | 0.67 ± 0.00 c | 0.22 ± 0.00 b |

Values in the same column of the same category with the different letters differ significantly (p < 0.05).

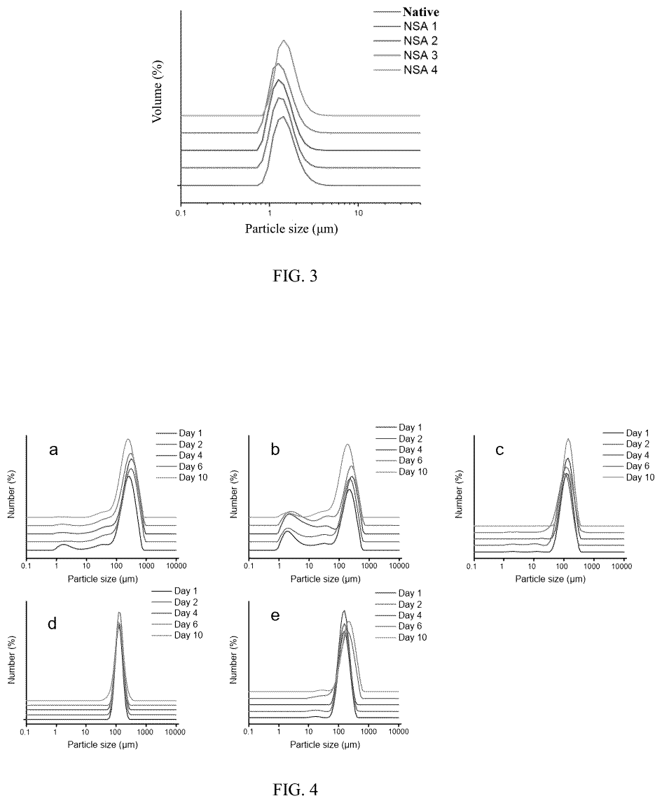

FIG. 3 shows the particle size distributions of unmodified and NSA modified quinoa starches. The D[4,3] of unmodified quinoa starch was 1.96 μm, consistent with previous studies and results obtained by SEM. As the degree of substitution increased, the particle size of quinoa starch first decreased and then increased (Table 1). Quinoa starch has granular aggregates in its unmodified state. Some aggregation of starch granules during preparation may cause overestimation of the particle size. NSA modification of starch imparted surface charge to the particles, which facilitated separation of particles and decreased the estimated mean diameter. The increased size of the highest substituted particles (NSA 4) may be due to disruption of starch granular structure caused by NSA, which promoted water ingress into the internal granular structure and increased the size. Surface gelatinization may also lead to granular swelling and fusion, which could be another reason for the increased diameter.

### Preparation of Pickering Emulsions

0.5 g of unmodified quinoa starch or NSA modified quinoa starch was suspended in 25 mL of 0.20 M NaCl aqueous solution. The slurry was homogenized at 10,000 rpm for 1 minute using a T25 digital Ultra-Turrax (IKA Works, Inc., Wilmington, USA). During mixing, 10 mL of rice bran oil containing Sudan red dye was slowly added to the slurry. The O/W Pickering emulsion was formed after homogenizing for another 2 minutes. The appearance, particle size analysis, emulsification capacity and stability were tested on days 1, 2, 4, 6 and 10 after preparation.

### Droplet Size and Stability of Emulsions

Photos of the Pickering emulsions were taken on day 1 (the first day of emulsion preparation), days 2, 4, 6 and 10. The total height of emulsion and aqueous phase were recorded, and the creaming index (CI) was calculated using the following formula:

$$CI = \frac{{H}_{S}}{{H}_{E}} \times 100 \%$$

where H_{S} is the height of serum phase, and H_{E} is the total height of emulsion.

TABLE 2
Creaming index of Pickering emulsions stabilized by unmodified
and NSA modified quinoa starches during storage

| Sample | Day 1(%) | Day 2(%) | Day 4(%) | Day 6(%) | Day 10(%) |
|---|---|---|---|---|---|
| Unmodified | 71.1 ± 3.6 a | 65.0 ± 1.2 a | 65.7 ± 1.9 a | 66.2 ± 0.8 a | 69.5 ± 1.5 a |
| NSA 1 | 64.1 ± 4.6 a | 61.2 ± 2.2 a | 61.0 ± 2.2 b | 61.2 ± 1.3 b | 61.3 ± 2.6 b |
| NSA 2 | 47.4 ± 2.1 b | 46.7 ± 2.9 b | 46.7 ± 0.1 d | 46.9 ± 3.4 d | 48.1 ± 3.7 c |
| NSA 3 | 36.2 ± 1.5 c | 38.5 ± 0.6 c | 38.9 ± 1.3 e | 39.3 ± 1.0 e | 37.9 ± 0.7 d |
| NSA 4 | 46.4 ± 2.2 b | 48.7 ± 2.7 b | 51.9 ± 1.6 c | 54.9 ± 1.5 c | 50.3 ± 2.4 c |

Values in the same column of the same category with the different letters differ significantly (p < 0.05).

FIG. 4 shows particle size distributions of droplets in Pickering emulsions stabilized by unmodified/NSA modified quinoa starch in the examples of the present disclosure during storage: a) unmodified quinoa starch; b) NSA 1 (DS 0.0080); c) NSA 2 (DS 0.0175); d) NSA 3 (DS 0.0359); e) NSA 4 (DS 0.0548).

The mass-moment mean diameter (D[4,3]) of Pickering emulsions stabilized by unmodified quinoa starch/NSA modified quinoa starch was measured using a method similar to that for measuring starch suspensions above. The results are as follows:

TABLE 3
D[4, 3] of Pickering emulsions stabilized by unmodified
and NSA modified quinoa starches during storage

| Sample | Day 1(μm) | Day 2(μm) | Day 4(μm) | Day 6(μm) | Day 10(μm) |
|---|---|---|---|---|---|
| Unmodified | 224 ± 7 a | 276 ± 3 a | 283 ± 10 a | 268 ± 4 a | 253 ± 18 a |
| NSA 1 | 145 ± 6 b | 160 ± 5 b | 155 ± 9 b | 169 ± 5 b | 180 ± 10 b |
| NSA 2 | 126 ± 9 c | 121 ± 8 c | 129 ± 3 c | 126 ± 4 c | 133 ± 4 c |
| NSA 3 | 119 ± 1 c | 127 ± 7 c | 122 ± 2 c | 123 ± 2 c | 123 ± 3 c |
| NSA 4 | 149 ± 6 b | 154 ± 7 b | 156 ± 12 b | 163 ± 10 b | 175 ± 14 b |

Values in the same column of the same category with the different letters differ significantly (p < 0.05).

Comparative Study of Starches Modified with OSA, NSA, and DDSA

It was found that at low degrees of substitution, the emulsification performance of NSA modification was significantly better than that of OSA, while at high degrees of substitution, the emulsification performance of OSA modification was better than that of NSA modification.

NSA modification has significant advantages over OSA at low degrees of substitution. Although the degree of substitution of OSA 1 (DS: 0.0113) was higher than that of NSA 1 (DS: 0.0080), the particle size (Day 1: 167.4 μm) of the Pickering emulsion formed by OSA modified starch was larger than that (Day 1: 145 μm) formed by NSA modified starch. The latter also had better stability (NSA Day 10: 180 μm; OSA Day 10: 239.1 μm). OSA 2 (DS: 0.0200) showed the same trend compared to NSA 2 (DS: 0.0175). The possible reasons for the above results are that NSA has stronger hydrophobicity than OSA.

For high degrees of substitution, the emulsion performance of OSA modified starch was better than NSA modified starch. The particle size of Pickering emulsion formed by NSA 3 (DS: 0.0359) modification was close to that by OSA 3 (DS: 0.0283) modification (NSA 3 Day 1: 119 μm; OSA 3 Day 1: 124.3 μm), while the particle size of Pickering emulsion formed by NSA 4 (DS: 0.0548) modification was significantly larger than that by OSA 4 (DS: 0.0427) modification (NSA 4 Day 1: 149 μm; OSA 4 Day 1: 74.2 μm). The possible reason is that NSA molecules have stronger hydrophobicity, which tends to cause aggregation at high degrees of substitution.

In summary, the optimal degree of substitution for NSA to form Pickering emulsions is lower while that for OSA is higher. Pickering emulsions formed by NSA modified starch at low degrees of substitution (DS<0.03) are significantly better than those by OSA modified starch. Chemically modified starch products with high degrees of substitution have not been suitable for the food industry due to safety reasons, for example, FDA regulations limit the degree of substitution of OSA modified starch added to foods as no more than 0.023. Although there is no standard yet for NSA modified starch, it can be expected that under the same degree of substitution limit, NSA modified starch has stronger emulsification capability and forms more stable Pickering emulsions. Therefore, NSA modified starch also has broader application prospects.

Comparing DDSA modified starch (degree of substitution 0.0077) with NSA modified starch (degree of substitution 0.0080), it was found that: the emulsion stabilized by DDSA modified starch had a smaller size (DDSA, Day 1: 58.9 μm; NSA, Day 1: 145 μm) right after preparation compared to NSA modified starch, but the stability provided by NSA modified starch was significantly better than DDSA modified starch. On day 10, the particle size of the DDSA stabilized emulsion was 136.9 μm, increased by 132% from day 1, while the particle size of the NSA stabilized emulsion on day 10 was 180 μm, only increased by 24% from day 1. Therefore, the stability of Pickering emulsion formed by NSA modified starch is significantly better than that by DDSA modified starch with the same degree of substitution.

In summary, NSA has stronger hydrophobicity than OSA, and therefore can achieve the same emulsification effect at lower degrees of substitution. Although NSA has lower hydrophobicity than DDSA and has lower emulsification effect at the same degree of substitution, the stability of Pickering emulsion formed by NSA modified starch is significantly better than that by DDSA modified starch at the same degree of substitution. In addition, because DDSA is in solid form at room temperature and has high hydrophobicity, it is difficult for the esterification reaction to reach the optimal degree of substitution range for emulsification. NSA can more easily obtain high degrees of substitution, thus reaching the optimal region for emulsification, which is also more conducive to application and production.

The above are merely exemplary embodiments of the present invention, and not intended to limit the protection scope of the present invention. The protection scope of the present invention is defined by the appended claims.

1. A nonenyl succinic anhydride modified starch, wherein the nonenyl succinic anhydride modified starch has degree of substitution no more than 0.10.

2. The nonenyl succinic anhydride modified starch according to claim 1, wherein said degree of substitution does not exceed 0.06.

3. The nonenyl succinic anhydride modified starch according to claim 2, wherein said degree of substitution does not exceed 0.023.

4. The nonenyl succinic anhydride modified starch according to claim 3, wherein said degree of substitution is between 0.0050 and 0.020.

5. The nonenyl succinic anhydride modified starch according to claim 1, wherein the nonenyl succinic anhydride modified starch has De Brouckere mean diameter D[4,3] less than or equal to 5.0 μm.

6. The nonenyl succinic anhydride modified starch according to claim 5, wherein the De Brouckere mean diameter D[4,3] is less than or equal to 3.0 μm.

7. The nonenyl succinic anhydride modified starch according to claim 6, wherein the De Brouckere mean diameter D[4,3] is greater than or equal to 0.50 μm and less than or equal to 2.0 μm.

8. The nonenyl succinic anhydride modified starch according to claim 1, wherein said starch is derived from barley, wheat, quinoa, oats, sorghum, corn, potato, sweet potato, cassava, pea, or banana.

9. The nonenyl succinic anhydride modified starch according to claim 8, wherein said starch is derived from quinoa.

10. An emulsifier comprising the nonenyl succinic anhydride modified starch according to claim 1.

11. The emulsifier according to claim 10, wherein the emulsifier is used in foods, cosmetics or pharmaceuticals.

12. A Pickering emulsion comprising the nonenyl succinic anhydride modified starch according to claim 1.

13. A method for preparing the nonenyl succinic anhydride modified starch of claim 1, comprising:
1) mixing starch with water to form a suspension;
2) slowly adding nonenyl succinic anhydride to the suspension in step 1) under stirring;
3) adding base or acid to control the pH of the suspension; and
4) obtaining the nonenyl succinic anhydride modified starch by centrifugation.

14. The method according to claim 13, wherein the nonenyl succinic anhydride is first mixed with a solvent to form a mixture before adding to the suspension in step 1).

15. The method according to claim 14, wherein said solvent is selected from water, ethanol, acetone or mixtures thereof.

16. The method according to claim 15, wherein said solvent is water, and a surfactant is added in the mixture of nonenyl succinic anhydride and the solvent.

17. The method according to claim 16, wherein said surfactant is selected from sodium dodecyl sulfate, sodium hexadecyl sulfate, sodium octadecyl sulfate or mixtures thereof.
